(19) United States
(12) Patent Application Publication
(10) Pub. No.: US 2026/0174425 A1
(43) Pub. Date: Jun. 25, 2026

(54) Suture Anchors and Methods of Tissue Repairs

(71) Applicant: Arthrex, Inc., Naples, FL (US)

(72) Inventors: Jason A. VALENTIN, Fort Myers, FL (US); Thomas DOONEY, JR., Naples, FL (US); Matthew R. HERRINGTON, Naples, FL (US); John A. SODEIKA, Naples, FL (US)

(73) Assignee: Arthrex, Inc., Naples, FL (US)

(21) Appl. No.: 19/426,342

(22) Filed: Dec. 19, 2025

Related U.S. Application Data
(60) Provisional application No. 63/738,004, filed on Dec. 23, 2024.

(51) Int. Cl. A61B 17/04; A61B 17/00

(52) U.S. Cl. CPC A61B 17/0401; A61B 2017/00862; A61B 2017/0464

(57) ABSTRACT

Surgical constructs and methods for surgical repairs are disclosed. A surgical construct is in the form of a soft suture anchor with a single flexible limb. The single suture limb may be passed through soft tissue and remains outside the bone for tensioning and/or alternative usage.

### CROSS-REFERENCE TO RELATED APPLICATIONS

The application claims priority to U.S. Provisional Patent Application No. 63/738,004 filed Dec. 23, 2024, the disclosure of which is hereby incorporated by reference in its entirety herein.

### BACKGROUND

The disclosure relates to the field of surgery and, more specifically, to anchor constructs and associated methods of tissue repairs.

### SUMMARY

Surgical systems, assemblies, and methods for fixation of soft tissue are disclosed.

A surgical construct may create a reinforced repair. A surgical construct may be made completely of suture to achieve fixation in tissue without a separate anchoring body and with increased fixation. The design enables fixation by deployment into tissue of an anchor into a small bone hole with a single suture limb remaining outside the bone for tensioning and/or alternative usage. A plurality of single limb anchors may be employed. The surgical construct may be employed in fixation of first tissue to second tissue, for example, fixation of soft tissue to bone. The fixation may be knotted or knotless.

A surgical construct may include one or more single-limb suture anchors. A flexible strand may consist of one end that may be secured into a small, independent tissue hole and another end provided as a flexible single limb. The single-limb suture anchor may be secured into tissue (for example, soft tissue or bone) and the single limb may be employed for additional surgical steps that may be knotted or knotless repairs.

Methods of tissue repairs are also disclosed. In some implementations, a surgical construct provides tissue fixation by providing a single-limb all-suture soft anchor that does not require a separate anchoring body or similar structure. The single-limb anchor may be employed by itself or in combination with additional single-limb anchors (or other fixation devices) in knotted and/or knotless tissue to tissue repairs, such as soft tissue to soft tissue repairs or soft tissue to bone repairs.

These and other features and advantages of this disclosure will become apparent and will be understood from the following detailed description of the various aspects of the disclosure taken in conjunction with the accompanying drawings.

### BRIEF DESCRIPTION OF THE DRAWINGS

FIG. 1 illustrates a surgical repair with a surgical construct.

FIG. 2 illustrates another surgical repair with a surgical construct.

FIG. 3 illustrates another surgical repair with a surgical construct.

FIG. 4 illustrates another surgical repair with a surgical construct.

FIG. 5 illustrates another surgical repair with a surgical construct.

FIG. 6 illustrates another surgical repair with a surgical construct.

FIG. 7 illustrates another surgical repair with a surgical construct.

FIG. 8 illustrates another surgical repair with a surgical construct.

FIG. 9 illustrates another surgical repair with a surgical construct.

FIG. 10 illustrates a rotator cuff repair with surgical constructs.

FIG. 11 illustrates a rotator cuff repair at a stage subsequent to that in FIG. 10.

FIG. 12 illustrates a rotator cuff repair at a stage subsequent to that in FIG. 11.

FIG. 13 illustrates another rotator cuff repair at a stage subsequent to that in FIG. 11.

### DETAILED DESCRIPTION

A surgical construct may create a tensionable, reinforced, self-locking repair.

A surgical anchor may be made completely of suture to achieve fixation in tissue (soft tissue, graft, bone, etc.) and without a separate anchoring body and with increased fixation. The design enables fixation by deployment into tissue with a single suture tail (single limb) remaining outside the tissue for tensioning and/or alternative usage. The surgical construct may be employed in fixation of first tissue to second tissue, for example, fixation of soft tissue to bone. The fixation may be knotted or knotless.

A surgical construct may include a tensionable construct in the form of a single-limb suture anchor. A single-limb suture anchor may include a body for positioning within a first tissue and a flexible strand end in the form of a flexible single limb. The single-limb suture anchor may be secured into tissue (for example, soft tissue, ligament, tendon, bone, graft, etc.) and the single limb may be employed for additional surgical steps that may be knotted or knotless repairs.

Methods of tissue repairs are also disclosed. In some implementations, a surgical construct provides tissue fixation by providing a single-limb all-suture soft anchor that does not require a separate anchoring body or similar structure. The single-limb anchor may be employed in knotted and/or knotless tissue to tissue repairs, such as soft tissue to soft tissue repairs, or soft tissue to bone repairs, in a simple and fast manner.

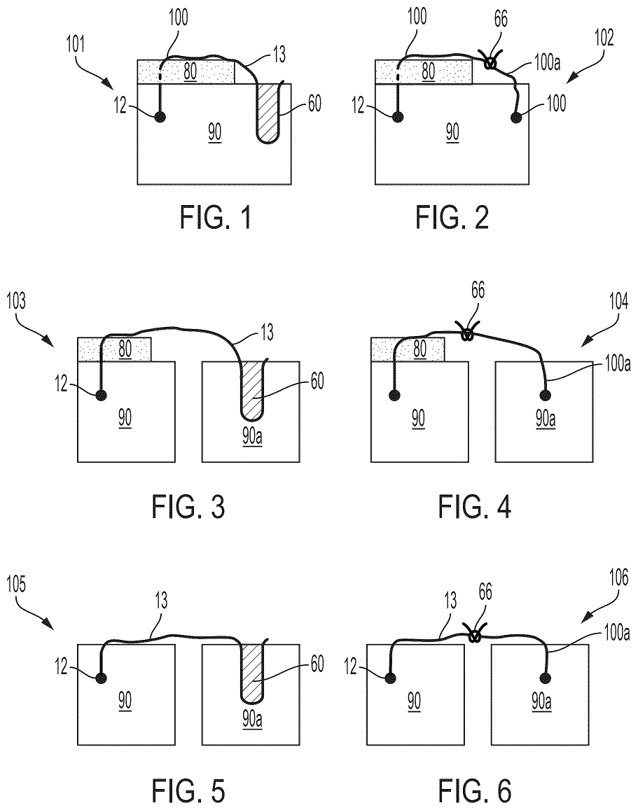

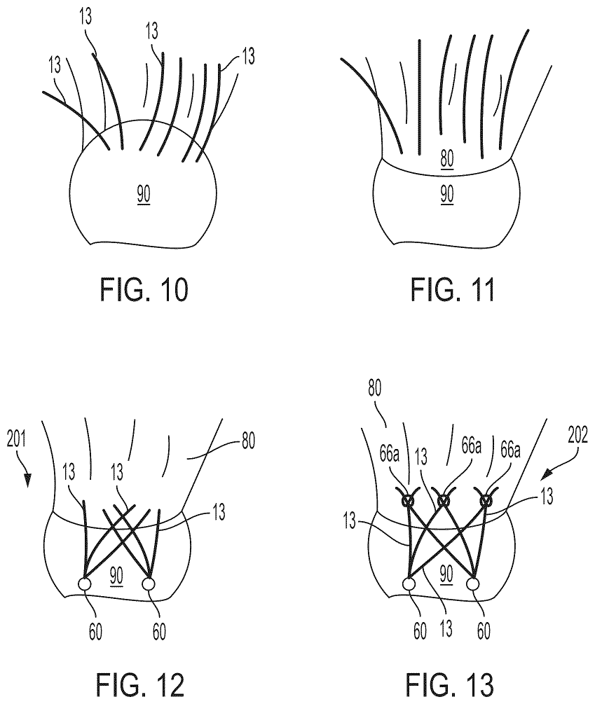

Referring now to the drawings, where like elements are designated by like reference numerals, FIGS. 1-9 illustrate exemplary surgical repairs 101, 102, 103, 104, 105, 106, 107, 108, 109 with surgical construct 100. FIGS. 10-13 illustrate exemplary rotator cuff repairs 201, 202 with surgical constructs 100.

Surgical construct 100 may be an implantable device; a suture anchor; an implant; a surgical assembly; a surgical system; a suture tape anchor; a tensionable, knotless construct; a tensionable, knotted construct; a single limb soft anchor; a single limb FiberTak® construct; or a SutureTape FiberTak® fixation device.

Implant 100 may be supported by soft or hard tissue and produces a single fixed repair limb for a custom tissue to tissue repair. The device may consist of a single flexible coupler with a first end that is configured in such a way as to create an anchoring body 12 or that bulks or bunches up when inserted into tissue (soft tissue or bone) and with a flexible end 13 (single repair limb 13). As detailed below, the single repair limb 13 may be used for additional procedures, for example, may be knotted with additional flexible coupler(s) and/or may be employed to be secured within different tissue locations with fixation devices such as knotted or knotless suture anchors.

Anchoring end 12 may be in the form of an anchor sheath or plurality of sheaths or similar anchoring structure(s) that allows it to be fixated into a small hole to create a point of fixation. Anchoring end 12 may include a single knot or multiple knots, or similar structures. Anchoring end 12 may include one or more static knots, for example. Anchoring end 12 may include square knots, sliding half-hitch knots or pre-tied knots such as a cerclage knot or a luggage-type knot, or combinations thereof. Additional knots may reinforce a first knot. The additional knot(s) lock the construct and prevent it from moving once inserted into tissue.

As detailed below, surgical construct 100 is an implantable device made completely of suture or sutures that achieves fixation in tissue without a separate anchoring body. The design enables for fixation by deployment into tissue with a single suture tail remaining outside the bone for tensioning and alternative usage.

FIG. 1 illustrates exemplary repair 101. Surgical construct 100 is positioned under first tissue 80 and within second tissue 90. First tissue 80 may be soft tissue, tendon, ligament, graft, etc. Second tissue 90 may be hard tissue such as bone. Surgical construct 100 attaches first tissue 80 to second tissue 90. Flexible limb 13 passes through first tissue 80 and may be fixated with a fixation device 60 into second tissue 90. Fixation device 60 may be an exemplary knotless anchor.

FIG. 2 illustrates exemplary repair 102. Surgical construct 100 is positioned under first tissue 80 and within second tissue 90. First tissue 80 may be soft tissue, tendon, ligament, graft, etc. Second tissue 90 may be hard tissue such as bone. Surgical construct 100 attaches first tissue 80 to second tissue 90. Flexible limb 13 passes through first tissue 80 and may be fixated with a knot 66 to another single limb anchor (such as construct 100) or a multi limb anchor 100a into bone 90.

FIG. 3 illustrates exemplary repair 103. Surgical construct 100 is positioned under first tissue 80 and within second tissue 90. First tissue 80 may be soft tissue, tendon, ligament, graft, etc. Second tissue 90 may be hard tissue such as bone. Surgical construct 100 attaches first tissue 80 to third tissue 90a. Third tissue 90a may be hard tissue such as bone. Flexible limb 13 passes through first tissue 80 and may be fixated with a fixation device 60 into third tissue 90a. Fixation device 60 may be an exemplary knotless anchor.

FIG. 4 illustrates exemplary repair 104. Surgical construct 100 is positioned under first tissue 80 and within second tissue 90. First tissue 80 may be soft tissue, tendon, ligament, graft, etc. Second tissue 90 may be hard tissue such as bone. Surgical construct 100 attaches first tissue 80 to third tissue 90a. Third tissue 90a may be hard tissue such as bone. Flexible limb 13 passes through first tissue 80 and may be fixated with a knot 66 to another single limb anchor (such as construct 100) or a multi limb anchor 100a into third tissue 90a (separate bone).

FIG. 5 illustrates exemplary repair 105. Surgical construct 100 is positioned in tissue 90. Tissue 90 may be hard tissue such as bone. Flexible limb 13 may be fixated with a fixation device 60 into a separate tissue 90a (another hard tissue such as bone). Fixation device 60 may be a knotless anchor.

FIG. 6 illustrates exemplary repair 106. Surgical construct 100 is positioned in tissue 90. Tissue 90 may be hard tissue such as bone. Flexible limb 13 may be fixated with a knot 66 to another single limb anchor (such as construct 100) or to a multi limb anchor 100a into a separate tissue 90a (another hard tissue such as bone).

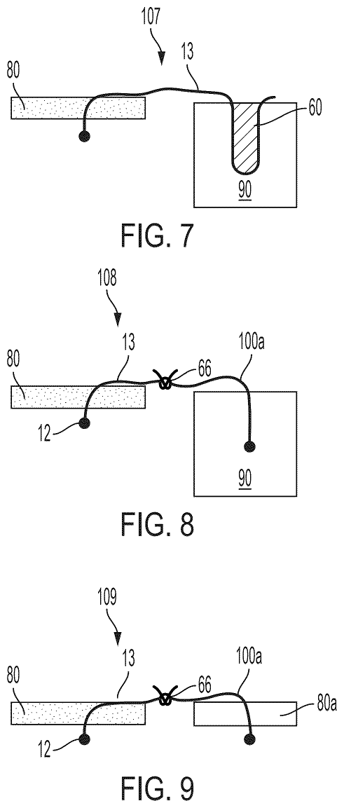

FIG. 7 illustrates exemplary repair 107. Surgical construct 100 is positioned in first tissue 80. First tissue 80 may be soft tissue, tendon, ligament, graft, etc. Flexible limb 13 may be fixated with a fixation device 60 into second tissue 90. Second tissue 90 may be hard tissue such as bone. Fixation device 60 may be a knotless anchor.

FIG. 8 illustrates exemplary repair 108. Surgical construct 100 is positioned in first tissue 80. First tissue 80 may be soft tissue, tendon, ligament, graft, etc. Flexible limb 13 may be fixated with a knot 66 to another single limb anchor (such as construct 100) or to a multi limb anchor 100a into a second tissue 90 (another hard tissue such as bone). Second tissue 90 may be hard tissue such as bone.

FIG. 9 illustrates exemplary repair 109. Surgical construct 100 is positioned in first tissue 80. First tissue 80 may be soft tissue, tendon, ligament, graft, etc. Flexible limb 13 may be fixated with a knot 66 to another single limb anchor (such as construct 100) or to a multi limb anchor 100a into a fourth tissue 80a (another soft tissue, tendon, ligament, graft, etc.).

Reference is now made to FIGS. 10-13 which illustrate exemplary rotator cuff repairs 201, 202 with single limb anchors.

FIG. 10 illustrates one or more anchors 100 inserted into bone 90 (first tissue 90). Exemplary anchors 100 may each have single suture limbs 13. FIG. 10 depicts six anchors 100 with six suture limbs 13; however, it must be understood that any number of anchors may be provided within tissue 90, depending on the extent of the repair and the surgeon's preferences.

FIG. 11 shows the sutures 13 passed through tissue 80 (second tissue 80) to be attached to bone 90; the sutures 13 may be passed individually or may be passed together. The anchors 100 may also be inserted directly through the tissue 80 into bone 90 (in a transtendon approach). In this implementation, the sutures 13 do not need to be passed through tissue 80.

FIG. 12 illustrates the sutures 13 fixated into bone 90 to fixate the soft tissue 80 against the bone 90, as part of repair 201. Three exemplary limbs 13 may be attached to one fixation device 60 and three remaining limbs 13 may be attached to another fixation device 60. Fixation device 60 may be a knotless anchor so that repair 201 is a knotless repair.

FIG. 13 illustrates repair 202. Sutures 13 may be tied once through the tissue 80 and then fixated into bone 90 to fixate the soft tissue 80 against the bone 90. In this exemplary implementation, two limbs 13 may be tied in single knot 66a to form three knots 66a on top of soft tissue 80. Strands 13 from each knot 66a may then be brought together and may be fixated within bone 90 with two fixation devices 60, as shown in FIG. 13.

Surgical construct 100 is a fixation device that acts as a soft anchor. The fixation device may be a simple round suture with ends formed also of round suture, of similar or different diameters. In some implementations, the fixation device may be a round suture or a suture tape. In some implementations, the fixation device may be a combination of suture and suture tape. In some implementations, the fixation device may be a suture tape terminating in round suture.

The fixation device may be a suture; suture tape; a braid; a repair suture; a round-flat suture; or a round-flat-round suture. The fixation device may be any flexible coupler and may be formed of various flexible materials and strands such as round suture, flat suture, ribbon, or flat tape (for example, suture tape) or combination of suture and tape. Exemplary materials may include suture, silk, cotton, nylon, polypropylene, polyethylene, ultrahigh molecular weight polyethylene (UHMWPE), polyethylene terephthalate (PET), and polyesters and copolymers thereof, or combinations thereof. The fixation device may be formed of a high strength suture material such as FiberWire® suture, sold by Arthrex, Inc. of Naples, Fla., and described in U.S. Pat. No. 6,716,234, the disclosure of which is incorporated by reference herein. FiberWire® suture is formed of an advanced, high-strength fiber material, namely ultrahigh molecular weight polyethylene (UHMWPE), sold under the tradenames Spectra® (Honeywell International Inc., Colonial Heights, Va.) and Dyneema® (DSM N.V., Heerlen, the Netherlands), braided with at least one other fiber, natural or synthetic, to form lengths of suture material. The fixation device may be braided or multi-filament suture such as FiberTape® suture tape (as disclosed in U.S. Pat. No. 7,892,256, the disclosure of which is incorporated in its entirety herewith). The fixation device may include elastic material. The fixation device may consist essentially of elastic tapered suture.

Fixation device 60 may be any implant, button, anchor, for example, knotted anchor, knotless anchor, or all-suture anchor, or any device that confers secure attachment and fixation of soft tissue 80 to bone 90. The fixation device 60 may be a knotless anchor such as a two-piece Arthrex PushLock® anchor, disclosed in U.S. Pat. No. 7,329,272, or an Arthrex SwiveLock® anchor, disclosed in U.S. Pat. Nos. 8,012,174 and 9,005,246, the disclosures of both of which are fully incorporated by reference in their entirety herein. The fixation device 60 may be an all-suture soft anchor. Details of an exemplary soft suture anchor with a soft anchor sleeve (sheath or tubular member) and flexible shuttling strands are set forth, for example, in U.S. Pat. No. 10,849,734 issued Dec. 1, 2020, entitled “Methods of Tissue Repairs,” the disclosure of which is incorporated by reference in its entirety herein.

The constructs, systems, and assemblies of the present disclosure may be employed in numerous soft tissue repairs and fixations, for example, fixation of soft tissue to bone.

A surgical construct 100 may be a fixation device with a first end 12 that forms an anchor body 12 and a single limb 13. Fixation device may be a soft suture anchor. First end 12 may be secured within a very small hole in tissue 90. Single limb 13 may be employed by itself or with additional single limbs 13 from additional single-limb constructs 100 in additional surgical steps such as soft tissue repairs. Ends 13 may be passed through and over soft tissue 80 and may be secured at a different location in bone 90, with one or more fixation devices 60. A plurality of constructs 100 may be secured within tissue 90. Single limbs 13 from different constructs 100 may be passed through and over soft tissue 80 to be secured to bone 90. Single limbs 13 may be passed separately and independently, or may be passed together with other limbs 13, in various configurations and patterns, to provide various types of repairs 101, 102, 103. 109, 201, 202. Surgical construct 100 may consist essentially of first end 12 and single limb 13.

Methods of soft tissue repair 101, 102, 103 . . . 109, 201, 202 which provide multiple points of fixation are also disclosed.

A method of tissue repair 101, 102, 103 . . . 109, 201, 202 may comprise inter alia the steps of: (i) securing a surgical construct 100 into a first tissue 90, the surgical construct being preformed with a suture body 12 that may be secured within a minimum diameter bone hole and with a single flexible limb 13; (ii) passing the single free limb 13 through and over a second tissue 80 to be positioned relative to the first tissue 90; and (iii) securing the single flexible limb 13 to the first tissue 90 with a fixation device 60. The fixation device 60 may be a knotted or knotless anchor. The surgical construct 100 may consist of body 12 and single limb 13.

A method of tissue repair 101, 102, 103 . . . 109, 201, 202 may comprise inter alia the steps of: (i) securing a plurality of surgical constructs 100 into a first tissue 90, each of the surgical constructs 100 being preformed with a suture body 12 that may be secured within a minimum diameter bone hole and with a single flexible limb 13; (ii) passing each of the single free limbs 13 through and over a second tissue 80 to be positioned relative to the first tissue 90; and (iii) securing the single flexible limbs 13 to the first tissue 90 with fixation devices 60. The fixation devices may be knotted or knotless anchors, or combinations thereof. Step (iii) may be conducted by (a) bringing together a first plurality of the single flexible limbs 13 and securing the first plurality of the single flexible limbs 13 to a first fixation device 60; and (b) bringing together a second plurality of the single flexible limbs 13 and securing the second plurality of the single flexible limbs 13 to a second fixation device 60. The method may further include tying together at least two of the single flexible limbs 13 into a knot after step (ii) and before step (iii). The tissue repair may be rotator cuff repair.

Various structural elements of surgical construct 100 may be visually coded, making identification and handling of the sheath and suture legs simpler. Easy identification of suture in situ is advantageous in surgical procedures, particularly during arthroscopic surgeries, endoscopic and laparoscopic procedures.

The surgical constructs of the present disclosure may be employed in endoscopic surgery. The term “endoscopic surgery” refers to surgical procedures within a patient's body through small openings as opposed to conventional open surgery through large incisions. Additionally, surgical constructs as disclosed herein may be utilized in other general surgical and specialty procedures such as soft tissue repairs.

The term “high strength suture” is defined as any elongated flexible member, the choice of material and size being dependent upon the particular application. For the purposes of illustration and without limitation, the term “suture” as used herein may be a cable, filament, thread, wire, fabric, or any other flexible member suitable for tissue fixation in the body.

What is claimed:

1. A soft anchor consisting of a flexible coupler with a first end and a second end, wherein the first end forms an anchoring body configured to be secured within a bone hole in bone, and wherein the second end is configured to secure soft tissue to the bone and to be secured within the bone.

2. The soft anchor of claim 1, wherein the flexible coupler is a suture or suture tape.

3. The soft anchor of claim 1, wherein the anchoring body is provided within a suture sheath.

4. The soft anchor of claim 1, wherein the anchoring body includes at least one knot.

5. The soft anchor of claim 1, wherein the flexible coupler consists essentially of elastic suture.

6. A soft tissue repair, comprising:
a plurality of soft anchors, each of the plurality of soft anchors including a flexible coupler with a first end and a second end, wherein the first end forms an anchoring body configured to be secured within a bone hole in bone, and wherein the second end is configured to secure soft tissue to the bone and to be secured within the bone; and
a plurality of fixation devices secured within the bone,
wherein second ends from the plurality of soft anchors are passed through the soft tissue and then brought together and secured to the bone with the plurality of fixation devices.

7. The soft tissue repair of claim 6, wherein the flexible coupler is elastic suture or elastic tape.

8. The soft tissue repair of claim 6, wherein the anchoring body includes at least one knot.

9. The soft tissue repair of claim 6, wherein the plurality of fixation devices includes at least one knotless anchor.

10. The soft tissue repair of claim 6, wherein the soft tissue is rotator cuff.

11. The soft tissue repair of claim 6, wherein the repair is a knotless repair.

12. The soft tissue repair of claim 6, wherein the repair is a knotted repair.

13. A method of tissue repair comprising:
securing, in a first tissue, at least one surgical construct consisting of a flexible coupler with a first end and a second end, wherein the first end forms an anchoring body configured to be secured within a hole in the first tissue, and wherein the second end is configured to secure a second tissue to the first tissue and to be secured within the first tissue;
passing the second end through and over the second tissue to be positioned relative to the first tissue; and
securing the second end to the first tissue with a fixation device.

14. The method of claim 13, wherein the first tissue is bone and the second tissue is tendon or ligament.

15. The method of claim 13, wherein the at least one surgical construct is an all-suture anchor formed of a single suture material.

16. The method of claim 13, wherein the fixation device is a knotless suture anchor.

17. The method of claim 13, wherein the tissue repair is a knotted repair or a knotless repair.

18. A method of tissue repair comprising:
securing, in a first tissue, a plurality of surgical constructs, each of the plurality of surgical constructs consisting of a flexible coupler with a first end and a second end, wherein the first end forms a body configured to be secured within a hole in the first tissue, and wherein the second end is configured to secure a second tissue to the first tissue and to be secured within the first tissue;
passing each of the second ends through and over the second tissue to be secured to the first tissue;
bringing together a first plurality of the second ends and securing the first plurality of the second ends to the first tissue with a first fixation device; and
bringing together a second plurality of the second ends and securing the second plurality of the second ends to the first tissue with a second fixation device.

19. The method of claim 18, wherein each of the first and second fixation devices is a hard-body suture anchor.

20. The method of claim 18, wherein each of the first and second fixation devices is a knotless anchor.

21. The method of claim 18 further comprising tying together the first plurality of the second ends before securing the first plurality of the second ends to the first tissue with the first fixation device.

22. The method of claim 18 further comprising tying together the second plurality of the second ends before securing the second plurality of the second ends to the first tissue with the second fixation device.
